(19) United States
(12) Patent Application Publication
(10) Pub. No.: US 2024/0102085 A1
(43) Pub. Date: Mar. 28, 2024

(54) EXPONENTIAL BASE-GREATER-THAN-BASE 2 NUCLEIC ACID AMPLIFICATION USING HIGH- AND LOW-Tm PRIMERS

(71) Applicant: Cepheid, Sunnyvale, CA (US)

(72) Inventor: Sergey G. Lokhov, Seattle, WA (US)

(73) Assignees: Cepheid, Sunnyvale, CA (US); Cepheid, Sunnyvale, CA (US)

(21) Appl. No.: 18/109,795

(22) Filed: Feb. 14, 2023

Related U.S. Application Data
(60) Provisional application No. 63/310,467, filed on Feb. 15, 2022.

(51) Int. Cl. C12Q 1/6851

(52) U.S. Cl. CPC C12Q 1/6851

(57) ABSTRACT

Described herein are methods and compositions that provide highly efficient nucleic acid amplification. The method employs pairs of primers that differ significantly in Tm and a novel temperature/time course characterized by a temperature pulse during denaturation that enables a high-Tm primer (but not a low-Tm primer) to anneal and prime the synthesis of an additional nucleic acid strand beyond the two strands synthesized in a cycle of classical PCR. In some embodiments, this allows a 3-fold or greater increase of amplification product for each amplification cycle and therefore increased sensitivity and speed over conventional PCR.

### CROSS-REFERENCE TO RELATED APPLICATIONS

This application claims the benefit of U.S. provisional application No. 63/310,467, filed Feb. 15, 2022, which is hereby incorporated by reference in its entirety.

### STATEMENT AS TO RIGHTS TO INVENTIONS MADE UNDER FEDERALLY SPONSORED RESEARCH AND DEVELOPMENT

Not applicable.

### INCORPORATION BY REFERENCE OF SEQUENCE LISTING PROVIDED AS AN XML FILE

This application contains references to nucleic acid sequences that have been submitted concurrently herewith as the sequence listing ST26 format XML file “CPHDP020WO_SL.xml”, file size 10,768 bytes, created on Mar. 27, 2023, which is incorporated by reference in its entirety pursuant to 37 C.F.R. § 1.52(e)(5).

### FIELD

The methods and compositions described herein relate generally to the area of nucleic acid amplification. In particular, described herein are methods and compositions for increasing amplification efficiency.

### BACKGROUND

A wide variety of nucleic acid amplification methods are available, and many have been employed in the implementation of sensitive diagnostic assays based on nucleic acid detection. Polymerase chain reaction (PCR) remains the most widely used DNA amplification and quantitation method. Nested PCR, a two-stage PCR, is used to increase the specificity and sensitivity of the PCR (U.S. Pat. No. 4,683,195). Nested primers for use in the PCR amplification are oligonucleotides having sequence complementary to a region on a target sequence between reverse and forward primer targeting sites. However, PCR in general has several limitations. PCR amplification can only achieve less than two-fold increase of the amount of target sequence at each cycle. It is still relatively slow. In addition, the sensitivity of this method is typically limited, making it difficult to detect target that may be present at only a few molecules in a single reaction.

Efforts to increase amplification sensitivity and efficiency, without sacrificing specificity, have included the use of unique primer sets to achieve greater-than-base 2 amplification (U.S. Pat. No. 10,273,534) and the incorporation of modified bases that pair preferentially with their unmodified complements in primers (U.S. Patent Pub. No. 2020/0239878).

Nucleic acid amplification typically includes multiple cycles of the sequential procedures of: annealing at least two primers with a complementary or substantially complementary sequences in a target nucleic acid; synthesizing at least one strand of nucleotides in a template-dependent manner using a polymerase; and separating the strands of the newly-formed nucleic acid duplex to generate template for subsequent cycles of strand synthesis. In standard PCR, each cycle produces one new nucleic acid duplex from a single target nucleic acid duplex, yielding two nucleic acid duplexes, and standard PCR is thus understood as “base 2” PCR. With unique primer designs, base 3, producing three duplexes per cycle, and base 6, producing 6 duplexes per cycle can be achieved.

Amplification can comprise thermocycling or can be performed isothermally. Isothermal amplification typically requires the use of a nucleic acid polymerase that has strand displacement activity and/or some other means to effect strand separation. Thermocycling is standardly carried out by subjecting a PCR reaction mixture to three temperatures per cycle in the following sequence: denaturation, usually at about 95° C.; annealing, usually at about 5° C. below the Tm of the primers; and extension (e.g., at about 72° C.). Some methods simplify this temperature/time course to two temperatures per cycle. For example, U.S. Pat. No. 9,428,781 describes “oscillating amplification” in which a two-temperature cycle includes an upper temperature and a lower temperature that differ by no more than 20° C. Regardless of the temperature/time course, primers are designed to have similar melting temperatures (Tms) so that both primers anneal under the same reaction conditions.

### SUMMARY

Described herein are methods and compositions based on the use of simple sets of two amplification primers per target nucleic acid that are expressly designed to have sufficiently different melting temperatures (Tms) that they anneal under different reaction conditions. They are used in an amplification method that entails a more complex temperature/time course per cycle than standard PCR. This simple, elegant method can be used to achieve greater-than-base 2 amplification for any target nucleic acid. Using a primer pair comprising a high-Tm primer and a low-Tm primer, base 3 amplification can be achieved by introducing a single high-Tm primer temperature pulse into the denaturation phase of the amplification temperature/time course to allow annealing and extension of the high-Tm primer, but not the low Tm primer. Introducing further such temperature pulses into the denaturation phase of the amplification temperature/time course further increases the base for exponential amplification. Thus, for example, two high-Tm primer temperature pulses can yield base 4 amplification.

Various embodiments contemplated herein may include, but need not be limited to, one or more of the following:

Embodiment 1: A method for amplifying a target nucleic acid in a sample to produce a target amplicon, the method comprising using a nucleic acid primer set comprising a high-Tm primer and a low-Tm primer, wherein: the target nucleic acid comprises a first template strand and, optionally, a second template strand, wherein the second template strand is complementary to the first template strand; the high-Tm primer is capable of annealing to the first template strand and priming an extension product from the high-Tm primer; the low-Tm primer is capable of annealing to the first extension product and priming an extension product from the low-Tm primer; wherein the high-Tm primer has a Tm that is a least 5° C. higher than that of the low-Tm primer.

Embodiment 2: The method of embodiment 1, wherein the high-Tm primer has a Tm that is at least 10° C. higher than that of the low-Tm primer.

Embodiment 3: The method of embodiment 1, wherein the high-Tm primer has a Tm that is at least 15° C. higher than that of the low-Tm primer.

Embodiment 4: The method of embodiment 1, wherein the high-Tm primer has a Tm that is at least 20° C. higher than that of the low-Tm primer.

Embodiment 5: The method of embodiment 1, wherein the high-Tm primer has a Tm that is at least 25° C. higher than that of the low-Tm primer.

Embodiment 6: The method of any preceding embodiment, wherein the method comprises: contacting the sample with a reaction mixture comprising the nucleic acid primer set; raising the reaction temperature to a denaturation temperature to denature nucleic acids in the sample; pulsing the reaction by lowering the reaction temperature to a high annealing and extension temperature suitable for producing an extension product from the high-Tm primer and then raising the reaction temperature to the denaturation temperature; lowering the reaction temperature to a low annealing and extension temperature suitable for producing an extension product from the low-Tm primer, wherein the high-Tm primer also anneals and produces a further extension product; wherein said pulse enables the production of an additional amplicon beyond the number of amplicons produced in a single amplification cycle carried out on a double-stranded template without said pulse.

Embodiment 7: The method of embodiment 6, wherein said pulse is performed at least once per amplification cycle.

Embodiment 8: The method of embodiment 6, wherein said pulse is performed at least twice per amplification cycle.

Embodiment 9: The method of embodiment 6, wherein said pulse is performed at least three times per amplification cycle.

Embodiment 10: The method of embodiment 6, wherein said pulse is performed at least four times per amplification cycle.

Embodiment 11: The method of any one of embodiments 6-10, wherein: the denaturation temperature comprises a temperature of between 90° C. and 100° C.; the high annealing and extension temperature comprises a temperature of between 75° C. and 85° C.; and the low annealing and extension temperature comprises a temperature of between 60° C. and 70° C.

Embodiment 12: The method of embodiment 11, wherein the method comprises conducting a plurality of amplification cycles wherein each amplification cycle comprises subjecting the reaction mixture to the following temperatures in order: the denaturation temperature of between 92° C. and 98° C.; the high annealing and extension temperature of between 75° C. and 83° C.; the denaturation temperature of between 92° C. and 98° C.; and the low annealing and extension temperature of between 60° C. and 66° C.

Embodiment 13: The method of any one of embodiments 6 to 12, wherein the reaction mixture is held at the denaturation temperature and high annealing and extension temperature for the same amount of time and at the low annealing and extension temperature for longer than this amount of time.

Embodiment 14: The method of embodiment 13, wherein each amplification cycle comprises, in the following sequence: denaturing at 94-96° C. for 3-6 seconds; high annealing and extension at 79-81° C. for 0.1-6 seconds; denaturing at 94-96° C. for 3-6 seconds; and low annealing and extension at 63-64° C. for 3-8 seconds.

Embodiment 15: The method of any preceding embodiment, wherein the target nucleic acid is amplified at the rate of at least 3^{number of cycles} during an exponential phase of amplification.

Embodiment 16: The method of embodiment 11, wherein said pulse is performed at least twice per amplification cycle, and the method comprises conducting a plurality of amplification cycles wherein each amplification cycle comprises subjecting the reaction mixture to the following temperatures in order: the denaturation temperature of between 92° C. and 98° C.; the high annealing and extension temperature of between 75° C. and 83° C.; the denaturation temperature of between 92° C. and 98° C.; the high annealing and extension temperature of between 75° C. and 83° C.; the denaturation temperature of between 92° C. and 98° C.; the low annealing and extension temperature comprises a temperature of between 60° C. and 66° C.

Embodiment 17: The method of embodiment 16, wherein the reaction mixture is held at the denaturation temperature and high annealing and extension temperature for the same amount of time and at the low annealing and extension temperature for twice this amount of time.

Embodiment 18: The method of embodiment 17, wherein each amplification cycle comprises, in the following sequence: denaturing at 94-96° C. for 3-6 seconds; high annealing and extension at 79-81° C. for 0.1-6 seconds; denaturing at 94-96° C. for 3-6 seconds; high annealing and extension at 79-81° C. for 0.1-6 seconds; denaturing at 94-96° C. for 3-6 seconds; and low annealing and elongation at 63-64° C. for 3-8 seconds.

Embodiment 19: The method of any preceding embodiment, wherein the method further comprises detection of the target amplicon using a detection probe.

Embodiment 20: The method of embodiment 19, wherein the Tm of the detection probe is between that of the high-Tm primer and the low-Tm primer.

Embodiment 21: The method of any preceding embodiment, wherein the nucleic acid amplification is carried out in multiplex using at least two nucleic acid primer sets for amplifying at least two target nucleic acids, each comprising a high-Tm primer and a low-Tm primer, wherein, for each nucleic acid primer set, the high-Tm primer has a Tm that is a least 5° C. higher than that of the low-Tm primer.

Embodiment 22: A nucleic acid primer set comprising the primers set forth in any one of embodiments 1-4 or 21.

Embodiment 23: The method of any one of embodiments 1 to 21 or the nucleic acid primer set of embodiment 22, wherein the high-Tm primer is longer than the low-Tm primer.

Embodiment 24: The method of any one of embodiments 1 to 21 or the nucleic acid primer set of embodiment 22, wherein the high-Tm primer is more G-C-rich, or contains more stabilizing bases, than the low-Tm primer.

Embodiment 25: The method of any one of embodiments 1 to 21 or the nucleic acid primer set of embodiment 22, wherein the low-Tm primer is less G-C-rich, or contains more destabilizing bases, than the high-Tm primer.

### BRIEF DESCRIPTION OF THE DRAWINGS

FIG. 1: A schematic drawing showing the relative melting temperatures (Tms) of an illustrative amplicon (the target nucleic acid being amplified), a probe used to detect the amplicon, and primers used for standard, base 2 PCR (“PCR\hat{ }2”). As shown, the Tins of the primers are within a few degrees of one another.

FIG. 2: A schematic drawing showing an illustrative embodiment of the primers and method described herein. This method employs primers with Tins that differ significantly; as shown, the Tm difference between the high-Tm primer and the low-Tm primer is at least 10° C.

FIG. 3: A schematic drawing showing an illustrative temperature/time course of a single cycle of one embodiment of the method described herein. This drawing shows a single temperature pulse inserted into the denaturation phase at 95° C. Before this pulse, denaturation has separated two strands of target nucleic acid duplex, as shown. During the temperature pulse, the temperature is reduced to an appropriate temperature for the high-Tm primer to bind and produce a new nucleic acid strand. In this illustration, the high-Tm primer is the “forward” primer (“F primer”), which produces a “sense” (or “top” or 5′-to-3′, reading left-to-right) strand, although the method works just as well with the high-Tm primer being the “reverse” primer (“R primer”), in which case this step would produce an “antisense” (or “bottom” or 3′-to-5′, reading left-to-right) strand. In either case, the pulse ends with a return to the denaturation temperature and three nucleic acid strands that can each serve as a template when the temperature is reduced to an appropriate temperature for the low-Tm primer to bind the available templates (here, two sense strands) and produce new nucleic acid strands. At this temperature, the high-Tm primer will also bind the available template (here, the antisense strand) to produce a new nucleic acid strand. As a result, a single cycle of amplification ends with 3 copies of initial, single nucleic acid duplex, resulting in base 3 PCR (“PCR\hat{ }3”).

FIG. 4: A schematic drawing showing an illustrative temperature/time course of a single cycle of one embodiment of the method described herein. This drawing shows a single temperature pulse inserted into the denaturation phase at 95° C., similar to FIG. 3. However, whereas FIG. 3 shows a pulse in which the temperature is held for a period of time at a temperature appropriate for the high-Tm primer to bind, the pulse in FIG. 4 is a spike down in temperature, where the temperature is not held at the temperature appropriate for the high-Tm primer to bind, but is instead immediately increased.

FIG. 5: PCR results from Example 1: Comparison of real time amplification curves of hgDNA β-globulin housekeeping gene sequence by PCR\hat{ }2 and PCR\hat{ }3 generated with TaqMan probe. The left-hand series curves are from PCR\hat{ }3 reactions, and the right-hand series of curves are from PCR\hat{ }2 reactions, demonstrating a 10-cycle reduction in Ct for PCR\hat{ }3 versus PCR\hat{ }2.

FIG. 6: PCR results from Example 2: Comparison of PCR\hat{ }2 with PCR\hat{ }3 and PCR\hat{ }4. The exponential portions of the curves are, from left to right: PCR\hat{ }4, PCR\hat{ }3, and PCR\hat{ }2. The results reproduce the 10-cycle reduction in Ct for PCR\hat{ }4 versus PCR\hat{ }2 and show a further 4-cycle reduction in Ct for PCR\hat{ }4 versus PCR\hat{ }3.

FIG. 7: Example 3: Comparison of real time amplification curves of an alternative hgDNA β-globulin housekeeping gene sequence by PCR\hat{ }2 and PCR\hat{ }3 generated with TaqMan probe in a reaction mixture including 5% (vol/vol) DMSO.

FIG. 8A-8B: Schematic representations of designs for convectively-driven thermal cyclers for use in PCR (8A) and an illustrative embodiment of the methods described herein (8B).

### DETAILED DESCRIPTION

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonuclotides sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleotide sequence or can be less than perfectly complementary. In some embodiments, the primer has at least 65% identity to the complement of the target nucleotide sequence over a sequence of at least 7 nucleotides, more typically over a sequence in the range of 10-30 nucleotides, and, in some embodiments, over a sequence of at least 14-25 nucleotides, and, in some embodiments, has at least 75% identity, at least 85% identity, at least 90% identity, or at least 95%, 96%, 97%, 98%, or 99% identity. It will be understood that certain bases (e.g., the 3′ base of a primer) are generally desirably perfectly complementary to corresponding bases of the target nucleotide sequence. Primer and probes typically anneal to the target sequence under stringent hybridization conditions.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/112579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18—(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation.

The naturally occurring bases adenine, thymine, uracil, guanine, and cytosine, which make up DNA and RNA, are described herein as “unmodified bases” or “unmodified forms.”

The term “modified base” is used herein to refer to a base that is not a canonical, naturally occurring base (e.g., adenine, cytosine, guanine, thymine, or uracil). Examples of modified bases are 2-thiothymine and 2-aminoadenine.

Nucleotides comprising modified bases are referred to herein as “modified nucleotides.”

As used herein, the “Tm of a primer” refers to the Tm (melting temperature) of a double-stranded form of the primer.

A “high-Tm primer” is defined herein relative to a “low-Tm primer” in a nucleic acid primer set. The “high-Tm primer” is characterized by a Tm that is higher than that of the low-Tm primer.

The terms “pulsing” and “pulse” are used herein with reference to a nucleic acid amplification reaction to indicate a step in which the reaction temperature, which is typically at the denaturation temperature at this point in temperature cycling, is (1) lowered to an annealing and extension temperatures that allows a high-Tm primer to anneal and be extended, and then (2) increased to the denaturation temperature. Those of skill in the art readily appreciate that denaturation can occur within a temperature range and that a return to “the” denaturation temperature need not be a return to the exact, same denaturation temperature of the reaction before the pulse began. The terms “pulsing” and “pulse” encompass a “spike down” in temperature, where the temperature is lowered to the annealing and extension temperature for the high-Tm primer and then immediately increased, as well as the situation in which the temperature is lowered to, and held at, the annealing and extension temperature for a, typically short, period of time.

All ranges described herein include their endpoints, unless otherwise indicated.

The notation “\hat{ }X” refers to the base for exponential nucleic acid amplification. Classical PCR is base 2 and therefore denoted as “PCR\hat{ }2.” PCR\hat{ }3 refers to base 3 PCR, PCR\hat{ }4 reference to base 4 PCR, and so on.

### Increasing Amplification Efficiency Using High-Tm and Low-Tm Primer Sets

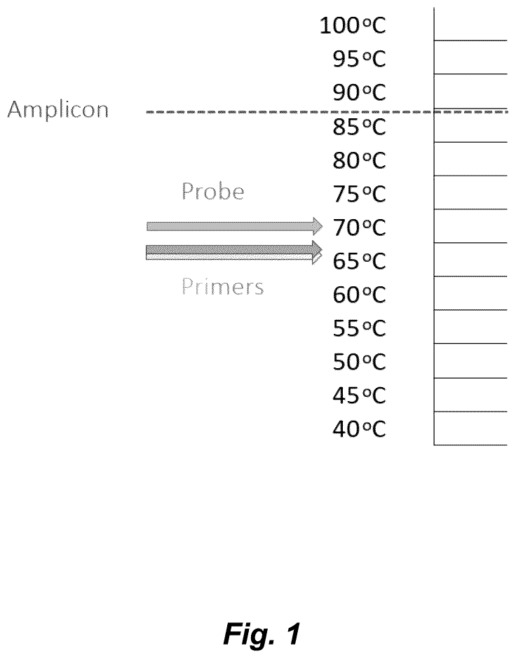

The methods described herein make use of nucleic acid primer sets that include one or more pairs of high- and low-Tm primers for each target nucleic acid to be amplified. This approach is diametrically opposed to that of classical PCR, for example, in which primers are designed to have Tins that are as close as possible. FIG. 1 shows a schematic illustration of a typical case of classical PCR, employing a probe, in which the Tins of the two primers is degrees apart, as indicated by the two close-together arrows pointing at just above 65° C. on the vertical temperature scale. The Tm of the probe is somewhat higher than that of both primers, at about 70° C.

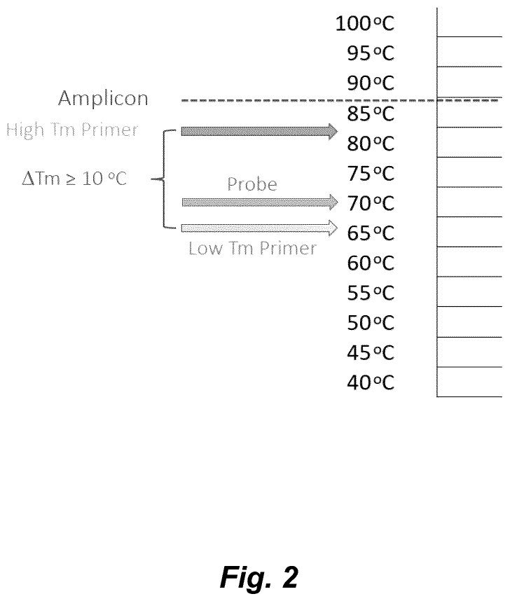

In contrast, the methods described herein make use of pairs of primers with significantly different Tins, as shown schematically in FIG. 2. In this illustrative embodiment, the high-Tm primer has a Tm at around 82° C., whereas the low-Tm primer has a Tm of just above 65° C. Unlike in the classical PCR shown in FIG. 1, the Tm of the probe, in this illustration, in in between that of the high-Tm primer and the low-Tm primer. In various embodiments, the Tm of the probe is at least about 1, 2, 3, 4, 5, 6, 7, 8, 9, or 10 or more degrees C. higher than the Tm of the low-Tm primer. In some embodiments the Tm difference can fall with a range of Tins bounded by any of these values (and including the endpoints). For example, the Tm difference can be at least 1-10, 2-9, 3-8, 4-7, or 5-6, etc.

In some embodiments, the difference in the Tins of the high-Tm primer and the low-Tm primer can be about: 5, 6, 7, 8, 9, 10, 11, 12, 13, 14, 15, 16, 17, 18, 19, 20, 21, 22, 23, 24, 25, 26, 27, 28, 29, or 30 or more degrees C. In some embodiments the Tm difference can fall with a range of Tins bounded by any of these values (and including the endpoints). For example, the Tm difference can be on the order of 6-28, 7-25, 8-22, 9-21, or 10-20, etc.

Classical PCR employs a “forward” primer, which primes the polymerization of a “sense” or “top” or 5′-to-3′ (reading left-to-right) nucleic acid strand. In classical PCR, a “reverse” primer primes the polymerization an “antisense” or “bottom” or 3′-to-5′ (reading left-to-right) nucleic acid strand. In some embodiments, the high- and low-Tm primer pairs described herein are pairs of “forward” and “reverse” primers. For example, the high-Tm primer can be the forward primer, and the low-Tm primer can be the reverse primer; or the low-Tm primer can be the forward primer, and the high-Tm primer can be the reverse primer.

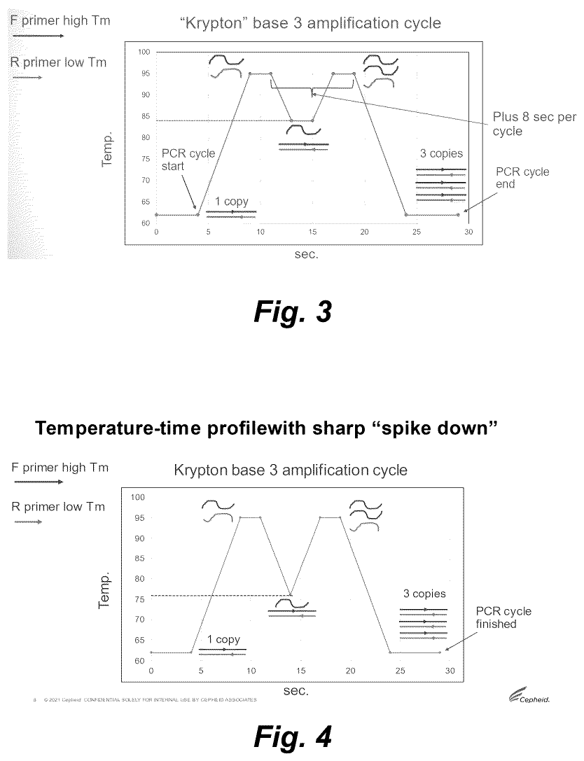

FIG. 3 shows an illustrative temperature/time course of a single cycle of one embodiment of the method described herein, in which the high-Tm primer is the forward primer and the low-Tm primer is the reverse primer. A novel feature of the present method is the temperature pulse shown as a dip in the center of the temperature/time course of FIG. 3. This temperature pulse and, in fact, the temperature/time course is a feature of the present method, regardless of whether the high-Tm primer is the forward primer or the reverse primer.

As shown in FIG. 3, the pulse facilitates the synthesis of a third nucleic acid strand from a starting double-stranded nucleic acid duplex. If the high-Tm primer is the forward primer, that third nucleic acid strand will be a sense strand, as shown in FIG. 3. If the high-Tm primer is the reverse primer, that third nucleic acid strand will be an antisense strand.

In either case, the pulse ends with a return to the denaturation temperature and three nucleic acid strands that can each serve as a template when the temperature is reduced to an appropriate temperature for the low-Tm primer to bind the available templates and produce new nucleic acid strands. At this temperature, the high-Tm primer will also bind the available template to produce a new nucleic acid strand. As a result, a single cycle of amplification ends with 3 copies of initial, single nucleic acid duplex, resulting in base 3 PCR (“PCR\hat{ }3”).

As shown in FIG. 3, if the high-Tm primer is the forward primer, during annealing/extension suitable for the low-Tm primer, the latter (which is the reverse primer) will anneal to the two sense strands generated during the pulse to generate two double-stranded duplexes, and the high-Tm primer will anneal to the antisense strand to generate a further double-stranded duplex.

If the high-Tm primer is the reverse primer, the pulse will have generated two antisense strands. During annealing/extension suitable for the low-Tm primer, the latter (which is the forward primer) will anneal to these two antisense strands to generate two double-stranded duplexes, and the high-Tm primer will anneal to the sense strand to generate a further double-stranded duplex.

The insertion of a single pulse into the denaturation phase of nucleic acid amplification increases the number of copies of present after one amplification cycle by one, thereby increase the base for exponential amplification by one. Classical PCR is characterized by exponential growth in amplicon copies with a base of 2. In one embodiment of the present method, the insertion of a single pulse into the denaturation phase of amplification increases the number of copies present after one amplification cycle to three, thus enabling exponential growth at base greater-than-2, and in theory up to at least base 3. In some embodiments, the method is carried out with 2, 3, or 4 more pulses, with the potential to increase the base for exponential amplification to base 4, base 5, or base 6, respectively. Further pulses can be carried out, if desired, although each pulse will increase the length of the denaturation phase of amplification, and the interest of not unduly extending the time for a single amplification cycle will, in many embodiments, make it advantageous to limit the number of pulses to those that provide the desired results in an appropriate timeframe for, e.g., an assay of interest.

In some embodiments, the pulse results in a drop in temperature of about: 5, 6, 7, 8, 9, 10, 11, 12, 13, 14, 15, 16, 17, 18, 19, 20, 21, 22, 23, 24, or 25 or more degrees C., and/or the drop in temperature falls within any range bounded by any of these values (and including the endpoints). For example, the temperature drop may fall within 6-24, 8-22, 10-20, 12-18, or 14-16 degrees C.

The duration of the temperature drop to the annealing/extension temperature for the high-Tm primer should be sufficient for extension from the high-Tm primer to generate a nucleic acid strand corresponding to the target sequence to be amplified. In some embodiments, the duration of this temperature drop is 0.1, 0.2, 0.3, 0.4, 0.5, 0.6, 0.7, 0.8, 0.9, 1, 2, 3, 4, 5, 6, 7, or 8 or more seconds, and/or this duration falls within any range bounded by any of these values (and including the endpoints). For example, the duration of this temperature drop may be 0.1-6, 0.2-5, 0.3-4, 0.5-3, 0.6-2, 0.7-1, 1-2, 2-7, 3-6, 3-5, or 3-4 seconds.

Samples

Nucleic acid-containing samples can be obtained from biological sources and prepared using conventional methods known in the art. In particular, nucleic acid-containing samples useful in the methods described herein can be obtained from any source, including unicellular organisms and higher organisms such as plants or non-human animals, e.g., canines, felines, equines, primates, and other non-human mammals, as well as humans. In some embodiments, samples may be obtained from an individual suspected of being, or known to be, infected with a pathogen (e.g., viral, bacterial, fungal or parasitic), an individual suspected of having, or known to have, a disease, such as cancer, or a pregnant individual.

Nucleic acids can be obtained from cells, bodily fluids (e.g., blood, a blood fraction, urine, etc.), or tissue samples by any of a variety of standard techniques. In some embodiments, the method employs samples of plasma, serum, spinal fluid, lymph fluid, peritoneal fluid, pleural fluid, oral fluid, and external sections of the skin; samples from the respiratory, intestinal genital, or urinary tracts; samples of tears, saliva, blood cells, stem cells, or tumors. Samples can be obtained from live or dead organisms or from in vitro cultures. Illustrative samples can include single cells, paraffin-embedded tissue samples, and needle biopsies. In some embodiments, the nucleic acids analyzed are obtained from a single cell.

Nucleic acids of interest can be isolated using methods well known in the art. The sample nucleic acids need not be in pure form, but are typically sufficiently pure to allow the steps of the methods described herein to be performed.

Target Nucleic Acids

Any target nucleic acid that can detected by nucleic acid amplification can be detected using the methods described herein. In typical embodiments, at least some nucleotide sequence information will be known for the target nucleic acids. For example, if the amplification reaction employed is PCR, sufficient sequence information is generally available for each end of a given target nucleic acid to permit design of suitable amplification primers.

The targets can include, for example, nucleic acids associated with pathogens, such as viruses, bacteria, protozoa, or fungi; RNAs, e.g., those for which over- or under-expression is indicative of disease, those that are expressed in a tissue- or developmental-specific manner; or those that are induced by particular stimuli; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

Primer Design

Primers suitable for nucleic acid amplification are sufficiently long to prime the synthesis of extension products in the presence of a suitable nucleic acid polymerase. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 to about 60 nucleotides, although it may contain more or fewer nucleotides. The primers should be sufficiently complementary to selectively anneal to their respective strands and form stable duplexes.

In general, one skilled in the art knows how to design suitable primers capable of amplifying a target nucleic acid of interest. For example, PCR primers can be designed by using any commercially available software or open source software, such as Primer3 (see, e.g., Rozen and Skaletsky (2000) Meth. Mol. Biol., 132: 365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

Adjustment of Primer or Probe Tm

Tm can be adjusted by adjusting the length of a sequence, the G-C content, and/or by including stabilizing or destabilizing base(s) in the sequence.

“Stabilizing bases” include, e.g., stretches of peptide nucleic acids (PNAs) that can be incorporated into DNA oligonucleotides to increase duplex stability. Locked nucleic acids (LNAs) and unlocked nucleic acids (UNAs) are analogues of RNA that can be easily incorporated into DNA oligonucleotides during solid-phase oligonucleotide synthesis, and respectively increase and decrease duplex stability. Suitable stabilizing bases also include modified DNA bases that increase the stability of base pairs (and therefore the duplex as a whole). These modified bases can be incorporated into oligonucleotides during solid-phase synthesis and offer a more predictable method of increasing DNA duplex stability. Examples include AP-dC (G-clamp) and 2,6-diaminoadenine, as well as 5-methylcytosine and C(5)-propynylcytosine (replacing cytosine), and C(5)-propynyluracil (replacing thymine).

“Destabilizing bases” are those that destabilize double-stranded DNA by virtue of forming less stable base pairs than the typical A-T and/or G-C base pairs. Inosine (I) is a destabilizing base because it pairs with cytosine (C), but an I-C base pair is less stable than a G-C base pair. This lower stability results from the fact that inosine is a purine that can make only two hydrogen bonds, compared to the three hydrogen bonds of a G-C base pair. Other destabilizing bases are known to, or readily identified by, those of skill in the art.

Primers may be prepared by any suitable method, including, for example, direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; the solid support method of U.S. Pat. No. 4,458,066 and the like, or can be provided from a commercial source. Primers may be purified by using a Sephadex column (Amersham Biosciences, Inc., Piscataway, NJ) or other methods known to those skilled in the art. Primer purification may improve the sensitivity of the methods described herein.

Polymerase

The disclosed methods make the use of a polymerase for amplification. Such polymerases are well known and those of skill in the readily select a suitable polymerase for a particular embodiment. In some embodiments, the polymerase is a DNA polymerase that either has or lacks a 5′ to 3′ exonuclease activity. In some embodiments, the polymerase is one that is adapted for a “hot start.” The most widely used polymerases are natural and mutant forms of Taq and Tth polymerases.

Illustrative polymerase concentrations range from about 20 to 200 units per reaction. In various embodiments, the polymerase concentration can be at least: 10, 20, 30, 40, 50, 60, 70, 80, 90, 100, 110, 120, 130, 140, 150, 160, 170, 180, 190, or 200 or more units per reaction. In some embodiments, the polymerase concentration falls within a range bounded by any of these values, e.g., 10-200, 10-150, 10-100, 10-50, 20-150, 20-100, 20-50, 50-200, 50-150, 50-100, 100-200, 100-150, etc. units per reaction.

Amplification

The primer sets described above are contacted with sample nucleic acids under conditions wherein the primers anneal to their template strands, if present. The desired nucleic acid amplification method is carried out using a suitable DNA polymerase.

For amplification using a two-primer set, as described above, with a single pulse, an amplification rate of about 3^{number of cycles} during the exponential phase of amplification can be achieved. In some embodiments, this base 3 amplification method can reduce the number of amplification cycles required to detect a target nucleic acid about 12% to about 42%. Examples 1-3 demonstrate that, in several illustrative embodiments, this method reduced the number of amplification cycles required to detect a target nucleic acid by about 37%.

For amplification using a two-primer set, as described above, with two pulses, an amplification rate of about 4^{number of cycles} during the exponential phase of amplification can be achieved. In some embodiments, this base 4 amplification method can reduce the number of amplification cycles required to detect a target nucleic acid about 27% to about 57%. Examples 1-3 demonstrate that, in several illustrative embodiments, this method reduced the number of amplification cycles required to detect a target nucleic acid by about 52%.

In some embodiments, the amplification step is performed using PCR. For running real-time PCR reactions, reaction mixtures generally contain an appropriate buffer, a source of magnesium ions (Mg²⁺) in the range of about 1 to about 10 mM, e.g., in the range of about 2 to about 8 mM, nucleotides, and optionally, detergents, and stabilizers. An example of one suitable buffer is TRIS buffer at a concentration of about 5 mM to about 85 mM, with a concentration of 10 mM to 30 mM being preferred. In one embodiment, the Tris buffer concentration is 20 mM in the reaction mix double-strength (2×) form. The reaction mix can have a pH range of from about 7.5 to about 9.0, with a pH range of about 8.0 to about 8.5 as typical. Concentration of nucleotides can be in the range of about 25 mM to about 1000 mM, typically in the range of about 100 mM to about 800 mM. Examples of dNTP concentrations are 100, 200, 300, 400, 500, 600, 700, and 800 mM. Detergents such as TWEEN 20, TRITON X 100, BRIJ and NONIDET P40 may also be included in the reaction mixture. Stabilizing agents such as dithiothreitol (DTT, Cleland's reagent) or mercaptoethanol may also be included. In addition, master mixes may optionally contain dUTP as well as uracil DNA glycosylase (uracil-N-glycosylase, UNG). A master mix is commercially available from Applied Biosystems, Foster City, CA, (TaqMan® Universal Master Mix, cat. nos. 4304437, 4318157, and 4326708). In some embodiments, the reaction mixture contains one or more amplification enhancers, such as, e.g., dimethyl sulfoxide (DMSO), bovine serum albumin (BSA), tetramethylsulfoxide (TMSO), acetamide, betaine, and the like. DMSO is a Tm-reducing agent and can be used to reduce amplicon Tm by about 3-4 degrees; consequently, the time required for the denaturation step can be reduced.

Labeling Strategies

Any suitable labeling strategy can be employed in the methods described herein. Where the reaction is analyzed for presence of a single amplification product, a universal detection probe can be employed in the amplification mixture. In particular embodiments, real-time PCR detection can be carried out using a universal qPCR probe. Suitable universal qPCR probes include double-stranded DNA-binding dyes, such as SYBR Green, Pico Green (Molecular Probes, Inc., Eugene, OR), Eva Green (Biotium), ethidium bromide, and the like (see Zhu et al., 1994, Anal. Chem. 66:1941-48).

In some embodiments, one or more target-specific qPCR probes (i.e., specific for a target nucleotide sequence to be detected) is employed in the amplification mixtures to detect amplification products. By judicious choice of labels, analyses can be conducted in which the different labels are excited and/or detected at different wavelengths in a single reaction (“multiplex detection”). See, e.g., Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992); and Linck et al. (2017) “A multiplex TaqMan qPCR assay for sensitive and rapid detection of phytoplasmas infecting Rubus species,” PLOS One 12(5).

In some embodiments, it may be convenient to include labels on one or more of the primers employed in in amplification mixture.

Cycling Probes

In some embodiments, a cycling probe can be used for detection and, optionally, quantification of target nucleic acids in the methods described herein. Cycling probes have been used for years as a way of amplifying signal in amplification assays. Cycling probes are described in, e.g., PCT Publication No. WO 89/09284, and U.S. Pat. Nos. 5,011,769 and 4,876,187, which are incorporated herein by reference for this description.

U.S. Pat. No. 5,763,181 describes the use of fluorescently labeled cycling probes to detect target nucleic acids. Generally, the disclosed method employs a fluorescently labeled oligonucleotide substrate containing a nucleotide sequence that is recognized by the enzyme that catalyzes the cleavage reaction. The oligonucleotide substrate can be DNA or RNA and can be single- or double-stranded. The oligonucleotide can be labeled with a single fluorescent label or with a fluorescent pair (donor and acceptor) on a single strand of DNA or RNA. The choice of single- or double-label can depend on the efficiency of the enzyme employed in the method of the invention. There is no limitation on the length of the oligonucleotide substrate, so long as the fluorescent probe is labeled sufficiently far (e.g., 6-7 nucleotides) away from the enzyme cleavage site. Examples of fluorophores commonly used in this method include fluorescein isothiocyanate, fluorescein amine, eosin, rhodamine, dansyl, and umbelliferone. Other fluorescent labels will be known to the skilled artisan. Some general guidance for designing sensitive fluorescently labeled polynucleotide probes can be found in Heller and Jablonski's U.S. Pat. No. 4,996,143. This patent discusses parameters that can be considered when designing fluorescent probes. The cycling probe cleavage reaction can be catalyzed by such enzymes as DNases, RNases, helicases, exonucleases, restriction endonucleases, or retroviral integrases. Other enzymes that effect nucleic acid cleavage are known to the skilled artisan and can be employed to cleave cycling probes having their cognate cleavage sites.

In some embodiments, one or more modified bases can be included in any of the probes described herein. The considerations discussed above regarding the use of stabilizing and/or modified bases in probes also applies to probes.

In some embodiments, it may be convenient to include labels on one or more of the primers employed in in amplification mixture.

Primer/Probe Concentrations

Primers and, if present, probes can be included in the amplification mixture at concentrations typical for the amplification method (e.g., PCR). In some embodiments, it is advantageous to include the primers at a relatively high concentration to encourage fast annealing/hybridization to facilitate rapid pulse and cycling. In some embodiments, the concentrations of the high-Tm and low-Tm primers are the same, e.g., at least 500, 550, 600, 650, 700, 750, 800, or 850 nM (for each primer). In some embodiments, the concentration of each primer fall within a range bounded by any of these values (including the endpoints), e.g., 550-850 nm, 600-850 nm, 650-850 nM, 700-850 nm, or 750-850 nm. In the Examples below, the primer concentrations were 800 nM.

Exemplary Automation and Systems

In some embodiments, a target nucleic acid is detected using an automated sample handling and/or analysis platform. In some embodiments, commercially available automated analysis platforms are utilized. For example, in some embodiments, the GeneXpert® system (Cepheid, Sunnyvale, CA) is utilized.

The methods described herein are illustrated for use with the GeneXpert system. Exemplary sample preparation and analysis methods are described below. However, the present invention is not limited to a particular detection method or analysis platform. One of skill in the art recognizes that any number of platforms and methods may be utilized.

The GeneXpert® utilizes a self-contained, single use cartridge. Sample extraction, amplification, and detection may all be carried out within this self-contained “laboratory in a cartridge” (available from Cepheid—see www.cepheid.com). One of skill in the art will recognize that the methods disclosed herein are suitable for use with other cartridge-based systems comprising a cartridge having a plurality of fluidly connected chambers housed within a single disposable cartridge body that provides for automated sample preparation, nucleic acid extraction, amplification, and detection. In some embodiments, the cartridge allows for storage of dried reagents and provides for automated fluidic movements, reagent rehydration, and mixing at the time of sample processing. In some embodiments, the cartridge comprises multiple fluidic pathways to prevent or limit bubble trapping and contamination, while allowing for thermal cycling and optical monitoring of reaction progress in a reaction chamber that extends from the body of the cartridge.

Components of the cartridge include, but are not limited to, processing chambers containing reagents, filters, and capture technologies useful to extract, purify, and amplify target nucleic acids. In some embodiments, a rotary valve enables fluid transfer from chamber to chamber which may contain nucleic acids lysis and filtration components. An optical window enables real-time optical detection. A reaction tube enables very rapid thermal cycling.

In some embodiments, the GeneXpert® system includes a plurality of modules for scalability. Each module is configured with fluid sample handling and analysis components.

After the sample is added to the cartridge, the sample is contacted with lysis buffer and released nucleic acid is bound to a nucleic acid-binding substrate such as a silica or glass substrate. The sample supernatant is then removed and the nucleic acid eluted in an elution buffer such as a Tris/EDTA buffer. The eluate may then be processed in the cartridge to detect target genes as described herein. In some embodiments, the eluate is used to reconstitute at least some of the reagents, which are present in the cartridge as lyophilized particles.

In some embodiments, PCR is used to amplify and detect the presence of one or more target nucleic acids. In some embodiments, the PCR uses Taq polymerase with hot start function, such as AptaTaq (Roche).

In some embodiments, an off-line centrifugation is used to improve assay results with samples with low cellular content. The sample, with or without the buffer added, is centrifuged and the supernatant removed. The pellet is then resuspended in a smaller volume of supernatant, buffer, or other liquid. The resuspended pellet is then added to a GeneXpert® cartridge as previously described.

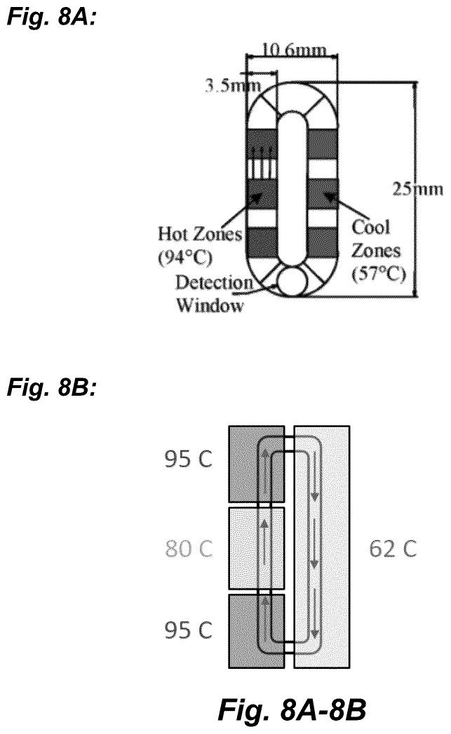

In some embodiments, a continuous, convection flow device can be employed in the method described herein. This device moves the reaction mixture through different temperature zones to achieve thermocycling. Convection PCR, and a suitable device for classical PCR, are described in Wheeler et al. (2004) “Convectively Driven Polymerase Chain Reaction Thermal Cycler,” 76:4011-4016. FIG. 8A shows an illustration of this device, and FIG. 8B shows how such a device can be designed for use in the method described herein.

Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

### Example 1: Comparison of Real Time Amplification Curves of hgDNA β-Globulin Housekeeping Gene Sequence by PCR\hat{ }2 and PCR\hat{ }3 Generated with TaqMan Probe

In this example, the oligonucleotide concentrations were 800 nM each in order to increase the rate of the hybridization reactions. The sequences of the forward and reverse primers, as well as of the probe, are listed in Table 1 (with Tm-stabilizing, modified nucleotides C* and A*), the PCR master mix components are shown in Table 2, and the temperature/time courses are shown in Table 3.

TABLE 1

| SEQ | | |
|---|---|---|
| ID | | Primer |
| No: | Sequence | Type |
| 1 | AAAACAGCATTCGCGCCGAGATGTCTC | Fwd |
| | GCTCCGTGGCC | |
| 2 | AAAAAAC*AGAAAGA*GA*GA*GTAG | Rev |
| 3 | FAM-CGCGAGCACAGCT*AAGG-BHQ1 | Probe |

Tm-stabilizing, modified nucleotides designated as C* and A* refer to 5-methylcytosine and 2,6-diaminoadenine, respectively. T* refers to a Tm-stabilizing, modified thymine. FAM and BHQ1 represent a Fluorescein amidite and a Black Hole quencher respectively.

TABLE 2

| Reagent | Concentration | |
|---|---|---|
| [Mg2+] | 5 | mM |
| [K+] | 60 | mM |
| dNTPs | 1 | mM total |
| Tris pH 8.5 | 20 | mM |
| BSA | 1 | mg/μL |
| TWEEN 20 | 0.3% | |
| hgDNA | 2E+3 | copies/reaction |
| APTATAQ (Roche) | 0.5 | U/μL |

TABLE 3

| | Temperature-time profile |
|---|---|
| PCR method (\hat{ }X) | (temp. in ° C./time in seconds) |
| PCR\hat{ }2 | 95/6-63/8 |
| PCR\hat{ }3 | 95/4-80/4-95/4-63/8 |

The PCR instrument used was PCRMAX ECO 48.

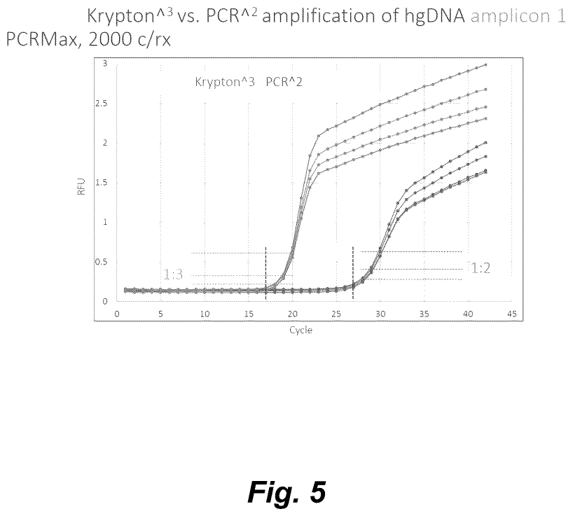

The results are shown in FIG. 5. PCR\hat{ }4 reduced the Ct by 10 cycles as compared to PCR\hat{ }2. Furthermore, in the beginning of the exponential phase amplification, the PCR\hat{ }3 curves for each new cycles increased the FAM signal by a factor of 3 versus the previous cycle. In the case of PCR\hat{ }2, the factor is only 2.

PCR\hat{ }3 generates about two-fold the End Point Fluorescence (EPF) versus PCR\hat{ }2. Without being bound by any particular theory, this phenomenon may be related to the reduction of competition between amplicon double-stranded complex formation and hybridization of primers and probe to a single-stranded amplicon. A higher-Tm primer can efficiently compete with double-stranded amplicon complex formation, which allows the lower-Tm primer and the probe hybridize to the complementary amplicon strand. Consequently, the reaction can generate a higher FAM signal.

### Example 2: Comparison of PCR\hat{ }2 with PCR\hat{ }3 and PCR\hat{ }4

For this example, the same oligonucleotides and reaction mixtures were used as in Example 1 (see Tables 1 and 2), and the results of temperature/courses with one (PCR\hat{ }3) or two (PCR\hat{ }4) pulses were compared. The temperature/time courses for each base of PCR is shown in Table 4.

TABLE 4

| | Temperature-time profile |
|---|---|
| PCR method (\hat{ }X) | (temp. in ° C./time in seconds) |
| PCR\hat{ }2 | 96/3-62/4 |
| PCR\hat{ }3 | 96/3-80/3-96/3-63/4 |
| PCR\hat{ }4 | 96/3-80/3-96/3- 80/3-96/3-63/4 |

The PCR instrument used was Cepheid GX with internal programming language GXterm.

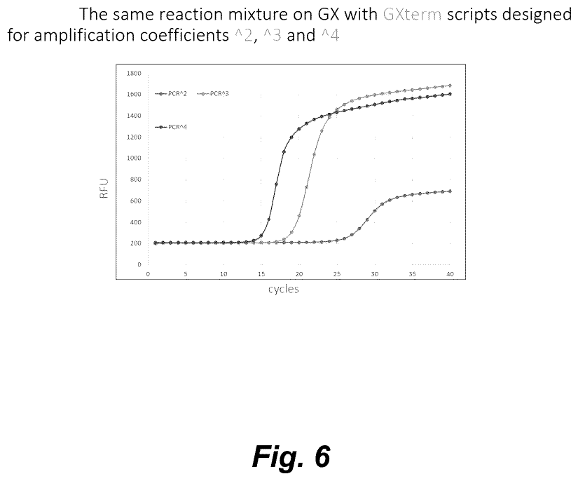

The results are shown in FIG. 6. PCR\hat{ }4 reduced the Ct by 4 cycles versus PCR\hat{ }3 and 14 cycles versus PCR\hat{ }2.

### Example 3: Comparison of Real Time Amplification Curves of an Alternative hgDNA β-Globulin Housekeeping Gene Sequence by PCR\hat{ }2 and PCR\hat{ }3 Generated with TaqMan Probe

PCR\hat{ }2 and PCR\hat{ }3 were carried on an alternative hgDNA β-globulin housekeeping gene sequence essentially as described above using the primer and probe designs (including Tm-stabilizing, modified nucleotides C* and A*) shown in Table 5. Oligonucleotide concentrations were 800 nM each to increase the rate of hybridization reactions. PCR master mix components were as shown in Table 2, but with the addition of 5% DMSO.

TABLE 5

| SEQ | | |
|---|---|---|
| ID | | Primer |
| No: | Sequence | Type |
| 4 | AAAACTGCGTGAGATTCTCCAGAGCA | Fwd |
| | AACTGGGCGGCATGGGCCCTGTGG | |
| 5 | AAAAAAGA*GCCAAAGA*GGAAG | Rev |
| 6 | FAM-CCCTCTGTAC*GAAAAGACC-BHQ1 | Probe |

Tm stabilizing modified nucleotides are designated as C* and A* refer to 5-methylcytosine and 2,6-diaminoadenine, respectively. FAM and BHQ1 represent a Fluorescein amidite and a Black Hole quencher respectively.

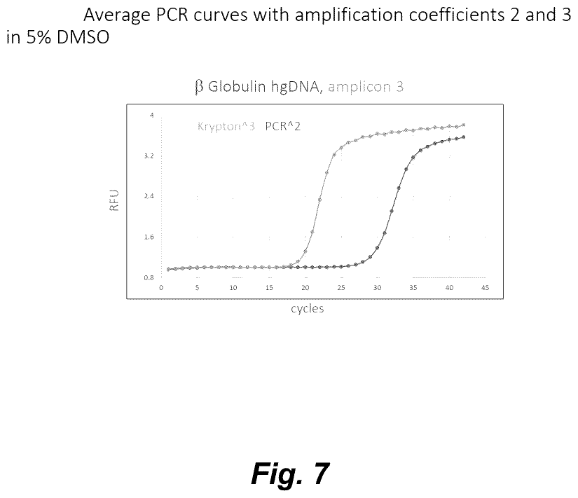

The results are shown in FIG. 7 where, as in Example 1, PCR\hat{ }3 reduced the Ct by 10 cycles versus PCR\hat{ }2 for the alternative hgDNA β-globulin housekeeping gene sequence target.

1. A method for amplifying a target nucleic acid in a sample to produce a target amplicon, the method comprising using a nucleic acid primer set comprising a high-Tm primer and a low-Tm primer, wherein:
the target nucleic acid comprises a first template strand and, optionally, a second template strand, wherein the second template strand is complementary to the first template strand;
the high-Tm primer is capable of annealing to the first template strand and priming an extension product from the high-Tm primer;
the low-Tm primer is capable of annealing to the first extension product and priming an extension product from the low-Tm primer;
wherein the high-Tm primer has a Tm that is a least 5° C. higher than that of the low-Tm primer.

2. The method of claim 1, wherein the high-Tm primer has a Tm that is at least 10° C. higher than that of the low-Tm primer.

3. The method of claim 1, wherein the high-Tm primer has a Tm that is at least 15° C. higher than that of the low-Tm primer.

4. The method of claim 1, wherein the high-Tm primer has a Tm that is at least 20° C. higher than that of the low-Tm primer.

5. The method of claim 1, wherein the high-Tm primer has a Tm that is at least 25° C. higher than that of the low-Tm primer.

6. The method of claim 1, wherein the method comprises:
contacting the sample with a reaction mixture comprising the nucleic acid primer set;
raising the reaction temperature to a denaturation temperature to denature nucleic acids in the sample;
pulsing the reaction by lowering the reaction temperature to a high annealing and extension temperature suitable for producing an extension product from the high-Tm primer and then raising the reaction temperature to the denaturation temperature;
lowering the reaction temperature to a low annealing and extension temperature suitable for producing an extension product from the low-Tm primer, wherein the high-Tm primer also anneals and produces a further extension product;
wherein said pulse enables the production of an additional amplicon beyond the number of amplicons produced in a single amplification cycle carried out on a double-stranded template without said pulse.

7. The method of claim 6, wherein said pulse is performed at least once per amplification cycle.

8. The method of claim 6, wherein said pulse is performed at least twice per amplification cycle.

9. The method of claim 6, wherein said pulse is performed at least three times per amplification cycle.

10. The method of claim 6, wherein said pulse is performed at least four times per amplification cycle.

11. The method of claim 1, wherein:
the denaturation temperature comprises a temperature of between 90° C. and 100° C.;
the high annealing and extension temperature comprises a temperature of between 75° C. and 85° C.; and
the low annealing and extension temperature comprises a temperature of between 60° C. and 70° C.

12. The method of claim 11, wherein the method comprises conducting a plurality of amplification cycles wherein each amplification cycle comprises subjecting the reaction mixture to the following temperatures in order:
the denaturation temperature of between 92° C. and 98° C.;
the high annealing and extension temperature of between 75° C. and 83° C.;
the denaturation temperature of between 92° C. and 98° C.; and
the low annealing and extension temperature of between 60° C. and 66° C.

13. The method of claim 6, wherein the reaction mixture is held at the denaturation temperature and high annealing and extension temperature for the same amount of time and at the low annealing and extension temperature for longer than this amount of time.

14. The method of claim 13, wherein each amplification cycle comprises, in the following sequence:
denaturing at 94-96° C. for 3-6 seconds;
high annealing and extension at 79-81° C. for 0.1-6 seconds;
denaturing at 94-96° C. for 3-6 seconds; and
low annealing and extension at 63-64° C. for 3-8 seconds.

15. The method of claim 11, wherein said pulse is performed at least twice per amplification cycle, and the method comprises conducting a plurality of amplification cycles wherein each amplification cycle comprises subjecting the reaction mixture to the following temperatures in order:
the denaturation temperature of between 92° C. and 98° C.;
the high annealing and extension temperature of between 75° C. and 83° C.;
the denaturation temperature of between 92° C. and 98° C.;
the high annealing and extension temperature of between 75° C. and 83° C.;
the denaturation temperature of between 92° C. and 98° C.;
the low annealing and extension temperature comprises a temperature of between 60° C. and 66° C.

16. The method of claim 15, wherein the reaction mixture is held at the denaturation temperature and high annealing and extension temperature for the same amount of time and at the low annealing and extension temperature for twice this amount of time.

17. The method of claim 15, wherein each amplification cycle comprises, in the following sequence:
denaturing at 94-96° C. for 3-6 seconds;
high annealing and extension at 79-81° C. for 0.1-6 seconds;
denaturing at 94-96° C. for 3-6 seconds;
high annealing and extension at 79-81° C. for 0.1-6 seconds;
denaturing at 94-96° C. for 3-6 seconds; and
low annealing and elongation at 63-64° C. for 3-8 seconds.

18. The method of claim 1, wherein the method further comprises detection of the target amplicon using a detection probe.

19. The method of claim 1, wherein the nucleic acid amplification is carried out in multiplex using at least two nucleic acid primer sets for amplifying at least two target nucleic acids, each comprising a high-Tm primer and a low-Tm primer, wherein, for each nucleic acid primer set, the high-Tm primer has a Tm that is a least 5° C. higher than that of the low-Tm primer.

20. A nucleic acid primer set comprising the primers set forth in claim 1.


SEQUENCE LISTING (6 sequences)

SEQ ID NO: 1 - DNA, 38 bp, synthetic construct
  source 1..38: mol_type = other DNA
aaaacagcattcgcgccgagatgtctcgctccgtggcc

SEQ ID NO: 2 - DNA, 22 bp, synthetic construct
  source 1..22: mol_type = other DNA
  modified_base 7: mod_base = OTHER; note = 5-methylcytosine
  modified_base 14: mod_base = OTHER; note = 2,6-diaminoadenine
  modified_base 16: mod_base = OTHER; note = 2,6-diaminoadenine
  modified_base 18: mod_base = OTHER; note = 2,6-diaminoadenine
aaaaaacagaaagagagagtag

SEQ ID NO: 3 - DNA, 17 bp, synthetic construct
  source 1..17: mol_type = other DNA
  modified_base 13: mod_base = OTHER; note = Tm-stabilizing modified thymine
cgcgagcacagctaagg

SEQ ID NO: 4 - DNA, 50 bp, synthetic construct
  source 1..50: mol_type = other DNA
aaaactgcgtgagattctccagagcaaactgggcggcatgggccctgtgg

SEQ ID NO: 5 - DNA, 21 bp, synthetic construct
  source 1..21: mol_type = other DNA
  modified_base 8: mod_base = OTHER; note = 2,6-diaminoadenine
  modified_base 16: mod_base = OTHER; note = 2,6-diaminoadenine
aaaaaagagccaaagaggaag

SEQ ID NO: 6 - DNA, 19 bp, synthetic construct
  source 1..19: mol_type = other DNA
  modified_base 10: mod_base = OTHER; note = 5-methylcytosine
ccctctgtacgaaaagacc